(19) United States
(12) Patent Application Publication
(10) Pub. No.: US 2025/0295864 A1
(43) Pub. Date: Sep. 25, 2025

(54) NEEDLE MECHANISM FOR AN AUTOMATED MEDICAMENT DELIVERY DEVICE

(71) Applicant: Insulet Corporation, Acton, MA (US)

(72) Inventors: Kyle Breingan, Westford, MA (US); Jeffrey Barnes, Medford, MA (US); Alexander Doudoumopoulos, Boxborough, MA (US)

(21) Appl. No.: 19/083,249

(22) Filed: Mar. 18, 2025

Related U.S. Application Data
(60) Provisional application No. 63/567,695, filed on Mar. 20, 2024.

(51) Int. Cl. A61M 5/32

(52) U.S. Cl. CPC A61M 5/3234

(57) ABSTRACT

Needle insertion mechanisms include arrangements of components, such as one or more of springs, actuator arms, or rails, that are configured to reduce and improve one or more of size or shape of the needle insertion mechanism and maintain the efficiency of the insertion and retraction actions thereof.

### CROSS-REFERENCE TO RELATED APPLICATION

This application claims the benefit under 35 U.S.C. § 119(e) of U.S. Provisional Patent Application Ser. No. 63/567,695, filed Mar. 20, 2024, the disclosure of which is hereby incorporated herein in its entirety by this reference.

### TECHNICAL FIELD

The present disclosure generally relates to automated medicament delivery devices. More particularly, the present disclosure relates to automated medicament delivery devices and cannula or needle insertion mechanisms for deploying a cannula used for medicament administration.

### BACKGROUND

Automated medicament delivery devices (e.g., Automated Insulin Delivery (AID) device, without limitation) are often used to administer medicaments to the user-body of a patient via a cannula inserted into the user-body to treat medical conditions.

The overall size of automated medicament delivery devices may influence the overall comfort to the user while being worn on the user-body. In particular, it is believed that the height is one of the more important dimensions to consider when reducing the overall volume of automated medicament delivery devices. For example, automated medicament delivery devices protruding away from the user-body may bump into or snag on clothing or other objects.

### BRIEF SUMMARY

Cannula or needle insertion mechanisms include arrangements of components, such as one or more of springs, actuator arms, or rails, that are configured to reduce and improve one or more of size or shape of the needle insertion mechanism and maintain the efficiency (e.g., speed, without limitation) of the insertion and retraction actions thereof.

In one illustrative embodiment, the present disclosure provides an automated medicament delivery device or system. The automated medicament delivery device or system including a cannula, a needle, and a cannula or needle insertion mechanism (hereafter referred to as a needle insertion mechanism for brevity). The cannula includes an insertion end. The needle includes a needle tip. The needle insertion mechanism includes an injection slide, a return slide, one or more rails, and an actuator. The injection slide configured to cause movement of the insertion end. The return slide configured to cause movement of the needle tip. The actuator configured to cause movement of the injection slide and the return slide along the one or more rails. The actuator including a spiral spring and a single actuator arm. The single actuator arm including a first end rotatably connected to the return slide and a second end rotatably connected to an external end of the spiral spring.

In another illustrative embodiment, the present disclosure provides an automated medicament delivery device or system. The automated medicament delivery device or system includes a cannula, a needle, and a needle insertion mechanism. The cannula including an insertion end. The needle including a needle tip. The needle insertion mechanism includes a single rail, an injection slide, a return slide, and an actuator. The single rail formed as a single unitary structure. The single rail including sides and a bend connecting the sides together defining a U-shape. The injection slide configured to move the insertion end therewith. The injection slide including an injection slide slot formed therein. The injection slide slot configured to receive at least one of the sides of the single rail. The return slide configured to move the needle tip therewith. The return slide including a return slide slot formed therein. The return slide slot configured to receive at least one of the sides of the single rail. The actuator configured to cause movement of the injection slide and the return slide along the single rail.

In a further illustrative embodiment, the present disclosure provides an automated medicament delivery device or system. The automated medicament delivery device or system includes a cannula, a needle, and a needle insertion mechanism. The cannula includes an insertion end. The needle includes a needle tip. The needle insertion mechanism includes a frame, an injection slide, and a return slide. The frame includes a vertical wall and a single rail. The vertical wall includes a drawn feature formed therein. The drawn feature defining a wall opening. The single rail formed as a single unitary structure. The single rail includes a first end received in the wall opening. The injection slide configured to move the insertion end therewith. The injection slide including an injection slide slot formed therein. The injection slide slot configured to receive the single rail. The return slide configured to move the needle tip therewith. The return slide including a return slide slot formed therein. The return slide slot configured to receive the single rail. The actuator configured to cause movement of the injection slide and the return slide along the single rail.

In another illustrative embodiment, the present disclosure provides an automated medicament delivery device or system. The automated medicament delivery device or system includes a cannula, a needle, and a needle insertion mechanism. The cannula includes an insertion end. The needle includes a needle tip. The needle insertion mechanism includes an injection slide, a return slide, a frame, and an actuator. The injection slide configured to move the insertion end therewith. The return slide configured to move the needle tip therewith. The frame includes one or more rails. The actuator includes a spring and an actuator arm. The actuator arm connected to the spring via a first hinge and to the injection slide via a second hinge.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a perspective view of an automated medicament delivery device with a needle injection mechanism in a pre-deployed state, in accordance with various embodiments.

FIG. 2 is a bottom perspective view of a portion of the automated medicament delivery device of FIG. 1, in accordance with various embodiments.

FIG. 3A is a side view of the needle injection mechanism of FIG. 1 and FIG. 2 in the pre-deployed state, in accordance with various embodiments.

FIG. 3B is a top view of the needle insertion mechanism of FIG. 1 and FIG. 2 in the pre-deployed state, in accordance with various embodiments.

FIG. 4A is a perspective view of the needle insertion mechanism of FIGS. 1-3 in a deployed state, in accordance with various embodiments.

FIG. 4B is a perspective view of the needle insertion mechanism of FIGS. 1-4A in a post-deployed state, in accordance with various embodiments.

FIG. 5A is a perspective view of an injection slide, a return slide, and a rail of a needle insertion mechanism, in accordance with various embodiments.

FIG. 5B is a top view of the injection slide, the return slide, and the rail of FIG. 5A, in accordance with various embodiments.

FIG. 5C is a cross-section of the injection slide of FIGS. 5A and 5B, in accordance with various embodiments.

FIG. 5D is a back view of the return slide of FIGS. 5A and 5B, in accordance with various embodiments.

FIG. 6A is a cross-section of various embodiments of an injection slide, a return slide, and a rail of a needle insertion mechanism, in accordance with various embodiments.

FIG. 6B is a top view of the injection slide of FIG. 6A, the return slide, and the rail, in accordance with various embodiments.

FIG. 6C is a cross-section of the injection slide of FIGS. 6A and 6B, in accordance with various embodiments.

FIG. 6D is a back view of the return slide of FIGS. 6A and 6B, in accordance with various embodiments.

FIG. 7A is a perspective view of a needle insertion mechanism in a pre-deployed state, in accordance with various embodiments.

FIG. 7B is a partial cross-section of the needle insertion mechanism of FIG. 7A, in accordance with various embodiments.

FIG. 8A is a perspective view of a frame of the needle insertion mechanism of FIG. 7A and FIG. 7B, in accordance with various embodiments.

FIG. 8B is a detailed cross-section of a portion of the frame of the needle insertion end of FIG. 8A, in accordance with various embodiments.

FIG. 8C is a detailed cross-section of a portion of the frame of the needle insertion mechanism of FIG. 8A, in accordance with various embodiments.

FIG. 9 is a cross-section of a portion of the needle insertion mechanism of FIG. 7A and FIG. 7B, in accordance with various embodiments.

FIG. 10 is a perspective view of a needle insertion mechanism, in accordance with various embodiments.

FIG. 11A is a top view of the needle insertion mechanism of FIG. 10 in a pre-deployed state, in accordance with various embodiments.

FIG. 11B is a top view of the needle insertion mechanism of FIG. 10 in a deployed state, in accordance with various embodiments.

FIG. 11C is a top view of the needle insertion mechanism of FIG. 10 in a partially retracted state, in accordance with various embodiments.

FIG. 11D is a top view of the needle insertion mechanism of FIG. 10 in a post-deployed/fully retracted state, in accordance with various embodiments.

### DETAILED DESCRIPTION

Needle insertion mechanisms for automated medicament delivery devices are discussed. Embodiments of such needle insertion mechanisms may allow a user to place an automated medicament delivery device on a person's skin and, via the action of the needle insertion mechanism, insert a soft cannula into the user-body utilizing a hard needle. Upon insertion, via the action of the needle insertion mechanism, the hard needle may be retracted, leaving only the soft cannula in the user-body for a comfortable wear. Insertion and retraction via the action of the needle insertion mechanism may be configured, as a non-limiting example, to occur in approximately 1/200^{th} of a second. Reducing the time duration of an insertion and retraction may, as non-limiting examples, reduce pain or user error.

As will be described below, various embodiments of a needle insertion mechanism herein include arrangements of components, such as one or more of springs, actuator arms, or rails, that are configured to reduce and improve one or more of size or shape of the needle insertion mechanism and maintain the efficiency (e.g., speed, without limitation) of the insertion and retraction actions thereof.

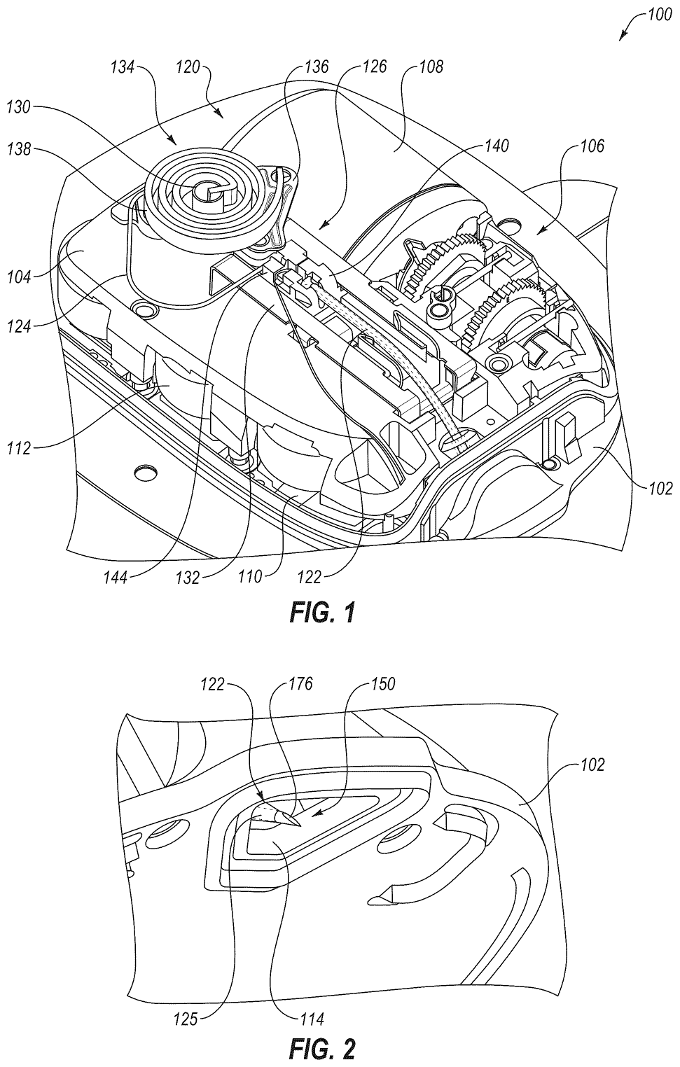

FIG. 1 is a perspective view of an automated medicament delivery device 100 with a needle insertion mechanism 120 in a pre-deployed state, in accordance with various embodiments. The automated medicament delivery device 100 is configured to administer a medicament into the user-body, such as subcutaneously into the user-body. In one or more embodiments, the automated medicament delivery device 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or subcombinations of one or more of the foregoing, without limitation.

Non-limiting examples of medicaments administrable by the automated medicament delivery device 100 include: insulin, glucagon-like peptide-1 receptor agonist (GLP-1), glucose-dependent insulinotropic polypeptide (GIP), or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

In various embodiments, the automated medicament delivery device 100 includes a housing 102, a chassis 104, a reservoir 108, a delivery mechanism 106, a printed circuit board 110 (“PCB 110”), a power source 112, and a needle insertion mechanism 120. The chassis 104 is configured to secure and position various components of the automated medicament delivery device 100 within the housing 102.

The reservoir 108 is configured to store and retain a medicament therein. As a non-limiting example, the reservoir 108 may be a hollow body, a chamber, a vial, without limitation. In various embodiments, the reservoir 108 is a fluid reservoir for holding medicament and may be, as a non-limiting example, formed from the walls of a cartridge. In the cartridge example, the chassis 104 may include a chamber (i.e., a space or region defined within chassis 104) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 108 will be greater in a pre-filled state than the volume in an exhausted state. Additionally or alternatively to the cartridge example, automated medicament delivery device 100 is a multi-part delivery device where one of the two parts includes the reservoir 108 and the other one of the two parts includes the PCB 110. The PCB 110 may include various electronic components including a controller. Either one of the two parts may optionally include delivery mechanism 106 (e.g., a pump mechanism, without limitation). The one of the two parts that includes reservoir 108 is disposable (i.e., a “disposable part”) and configured to be removably secured to the other part of automated medicament delivery device 100. When reservoir 108 is exhausted, the disposable part may be removed and a replacement part including a reservoir 108 optionally in a pre-filled state.

The delivery mechanism 106 is configured to urge fluid in the reservoir 108 toward the cannula 122 (described in further detail below) via tubing 124. Tubing 124 may be a separate flexible element or may comprise a proximal portion of needle 150. In various embodiments, the delivery mechanism 106 may be positioned adjacent to reservoir 108. The delivery mechanism 106 is configured to cause an amount of the medicament to be administered to the user-body by causing the amount to flow from the reservoir 108 toward and into the user-body via the cannula 122, which is in fluidic communication with the reservoir 108, such as via tubing 124. In various embodiments, the delivery mechanism 106 may utilize any suitable mechanism to generate positive displacement or negative displacement to transfer amounts of medicament from the reservoir 108 toward the cannula 122 and a user-body. Non-limiting examples of mechanisms include a ratchet gear pump, peristaltic pump, linear peristaltic pump, piston pump, gear pump, bellows pump, or diaphragm pump.

For example, the delivery mechanism 106 may apply a force to an urging mechanism (e.g., a plunger, flexible-walled tube, without limitation) free to move within the reservoir 108, and via such a force, move the urging mechanism in a direction that urges fluid in the reservoir 108 toward the aforementioned interface. In one or more examples, the delivery mechanism 106 may include an electrical motor (e.g., an AC or DC motor) that produces a force to, directly or indirectly, move the urging mechanism to perform a delivery action. A delivery action dispenses at a predetermined rate (i.e., a predictable amount of fluid over a predictable duration of time). The delivery mechanism 106 may be capable of multiple rates of delivery, and in one or more examples, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal may be determined and generated by a controller to correspond to a delivery action. A control signal may also be referred to herein as a “command” or an “instruction.” The controller may generate control signals corresponding to one or more delivery actions and may also generate a control signal that causes the delivery mechanism 106 to actuate.

The power source 112 provides power to the PCB 110, the various electronic components thereof, and the delivery mechanism 106.

FIG. 2 is a bottom perspective view of a portion of the automated medicament delivery device 100 of FIG. 1. Referring to FIG. 2, the housing 102 defines an opening 114 through which the cannula 122, and in particular, an insertion end 125 of the cannula 122, extends through during the insertion process and while positioned for administration of the medicament to the user-body. The needle 150, and in particular, a needle tip 176 of the needle 150, may guide the insertion end 125 into the user-body and may be withdrawn from the user-body once the insertion end 125 is positioned for administration of the medicament to the user-body. In various embodiments, the needle 150 is connected to and extends from the tubing 124; alternatively, tubing 124 may comprise a proximal portion of needle 150.

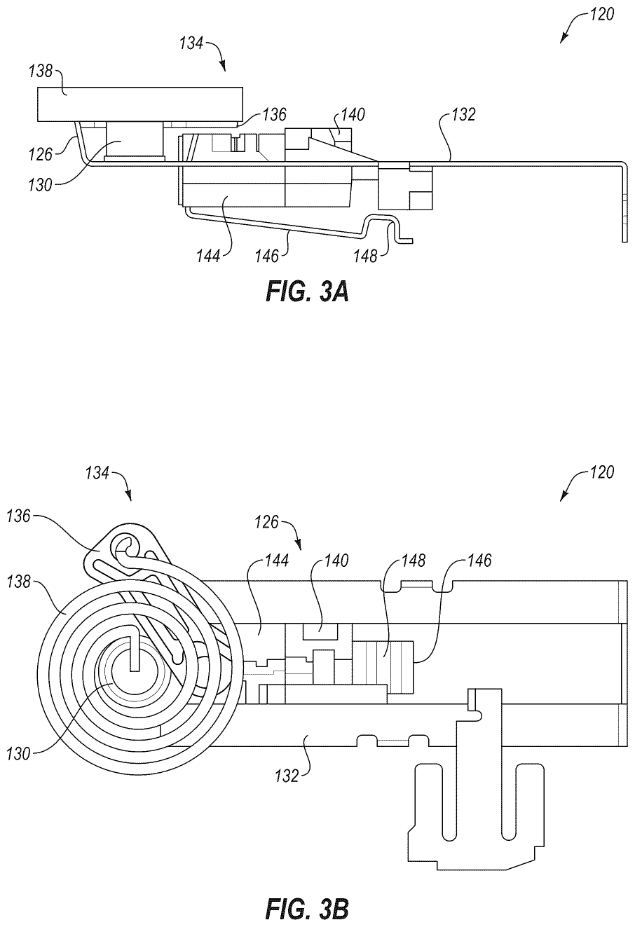

FIG. 3A is a side view of the needle insertion mechanism 120 of FIG. 1 in the pre-deployed state. FIG. 3A is a side view of the needle insertion mechanism 120 of FIG. 1 in the pre-deployed state. Referring to FIG. 1, FIG. 3A, and FIG. 3B, the needle insertion mechanism 120 may be mounted to the chassis 104 and is configured to insert the cannula 122 into the user-body (e.g., position an end of the cannula 122 in a subcutaneous position for subcutaneous administration of medicament into the user-body). In various embodiments, the needle insertion mechanism 120 includes a frame 126, an injection slide 140, a return slide 144, and an actuator 134. The frame 126 is configured to support the various components of the needle insertion mechanism 120. In various embodiments, the frame 126 includes a spring mount 130, one or more rails 132, and a locking arm 146. The spring mount 130 is configured to receive at least a portion of the actuator 134.

As will be described in further detail below, the one or more rails 132 is configured to guide actuation of the needle insertion mechanism 120, and in particular, maintain a controlled travel path for the injection slide 140 and the return slide 144. In the various embodiments illustrated in FIG. 1, FIG. 3A, and FIG. 3B, the frame 126 includes two rails 132.

The locking arm 146 includes a locking feature 148 configured to retain the injection slide 140 and the return slide 144 in the pre-deployed state.

The injection slide 140 is configured to cause the cannula 122 to move relative to the frame 126. In various embodiments, an end of the cannula 122 is secured to the injection slide 140 (e.g., received and secured in a slot formed in the injection slide 140, without limitation) and lateral movement of the injection slide 140 along the one or more rails 132 causes movement of the cannula 122.

The return slide 144 is configured to cause the needle 150 to move relative to the frame 126, and lateral movement along the one or more rails 132 causes movement of the needle 150. In various embodiments, a portion of the tubing 124 is secured to and configured to move with the return slide 144 (e.g., secured within a slot formed within the return slide 144, without limitation). In these various embodiments, the tubing 124 extends from the return slide 144 and into the cannula 122. The needle 150 may extend from an end of the tubing 124 or may be one and the same with the tubing 124. Needle 150 may be positioned within a lumen of cannula 122 and may include a tip protruding from an end of the cannula 122 (e.g., the end opposite the end secured to the injection slide 140, without limitation).

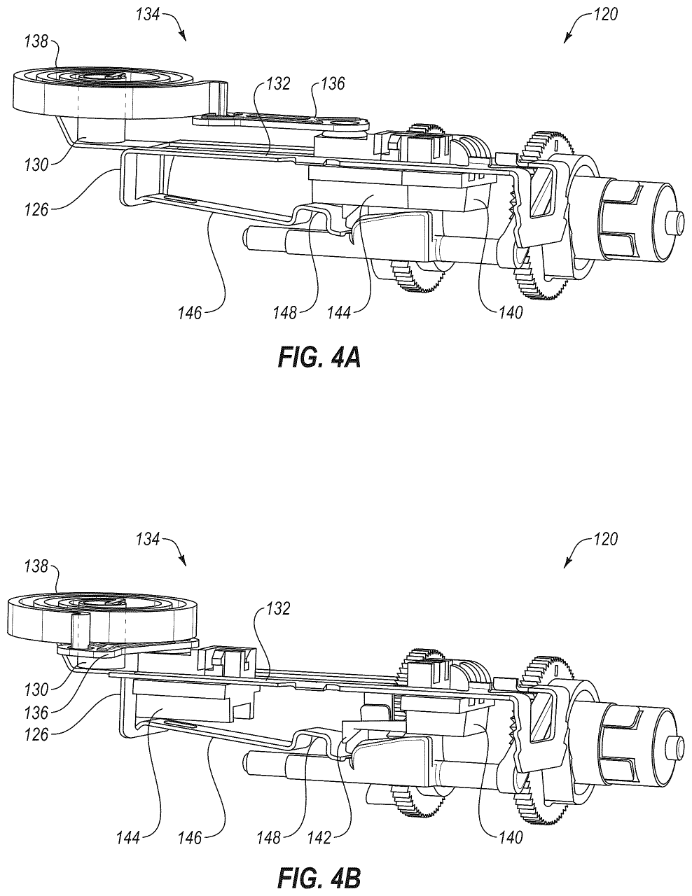

FIG. 4A is a perspective view of the needle insertion mechanism 120 of FIGS. 1-3 in a deployed state. FIG. 4B is a perspective view of the needle insertion mechanism 120 of FIGS. 1-4 in a post-deployed state. Referring to FIG. 1, FIG. 3A, FIG. 3B, FIG. 4A, and FIG. 4B, the actuator 134 is configured to cause both the return slide 144 and the injection slide 140 in a first direction, from a pre-deployed state to a deployed state along the one or more rails 132, resulting in injection of ends of the needle 150 and cannula 122 being inserted into the user-body (e.g., positioning an end of the cannula 122 for subcutaneous administration of the medicament, without limitation). Upon the return slide 144 and the injection slide 140 reaching the deployed state, the actuator 134 is configured to retract the return slide 144 from the deployed state to a post-deployed state in a second direction, opposite the first direction and away from the injection slide 140 resulting in retraction of the end of the needle 150 from the user-body and to a retracted position (e.g., a position within a portion of the cannula 122 exterior of the frame body 128). In various embodiments, the injection slide 140 includes a slide arm 142 that is configured to secure the injection slide 140 in the deployed state to ensure that the cannula 122 remains positioned for administration of the medicament during and after the return slide 144 is retracted to the post-deployed state (e.g., the slide arm 142 catches on the locking feature 148 of the locking arm 146, without limitation).

The actuator 134 includes a spring 138 and one or more actuator arms 136. In various embodiments, the spring 138 includes a spiral spring and a single actuator arm 136. An internal portion of the spiral spring is connected to the spring mount 130, and an external portion of the spiral spring is connected to an end of the actuator arm 136. In these various embodiments, the actuator arm 136 includes a first end rotatably connected to the return slide 144 and a second end rotatably connected to the spring 138, the second end opposite and distal to the first end. The spiral spring is configured to exert a force on the actuator arm 136 (e.g., a combination of rotational and translational force resulting from unwinding thereof, without limitation) causing movement of the actuator arm 136 (e.g., translation and rotation of the actuator arm 136, without limitation). In various embodiments, initial uncoiling of the spiral spring results in movement of the return slide 144 and the injection slide 140 in the first direction. The return slide 144 and the injection slide 140 may reach the deployed state. In various embodiments, further uncoiling of the spiral spring results in movement of the return slide 144 in the second direction, away from the injection slide 140, and resulting in withdrawal of the needle 150 from the user-body. In these embodiments, an end of the spiral spring connected to the actuator arm 136 while the spring 138 is in the wound condition is radially offset relative to a radial position that is closest to the insertion end 125 of the cannula 122. During the initial uncoiling the end of the spiral spring moves radially (in a first radial direction) and toward (and may move to) the radial position closest to the insertion end 125 to cause the return slide 144 and the injection slide 140 to reach the deployed state. During the further uncoiling of the spiral spring, the end of the spiral spring moves radially (in the first radial direction) away from the radial position closest to the insertion end 125 to a final uncoiled position. The initial uncoiling and the further uncoiling may be a single constant uncoiling of the spiral spring. The initial position of the end of the spiral spring and the final uncoiled position of the end may be about radially equidistant from the radial position closest to the insertion end 125. In some of these embodiments, the initial position of the end of the spring is about 180 degrees from the final uncoiled position of the end of the spring, and the radial position closest to the insertion end 125 is about 90 degrees from each of the initial position and the final uncoiled position.

The spiral spring may generally include a flat, loosely wound shape, which may reduce a height of the automated medicament delivery device 100, without requiring an increase in other dimensions thereof. Further, due to the general shape of the spiral spring, the needle insertion mechanism 120 may not require a second actuator arm 136 that may be required in other configurations, reducing complexity and weight of the automated medicament delivery device 100.

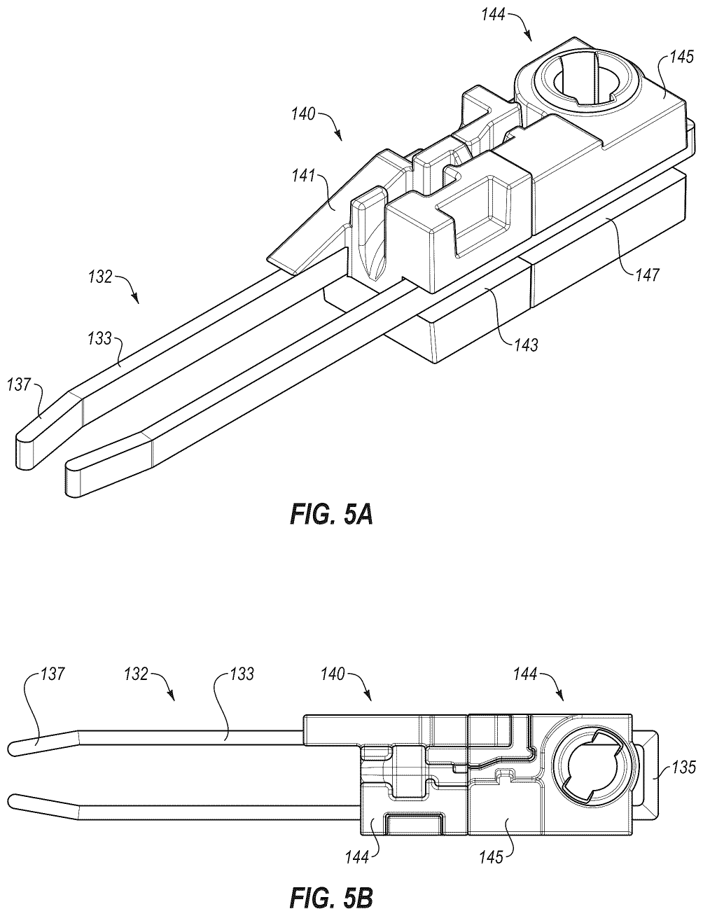

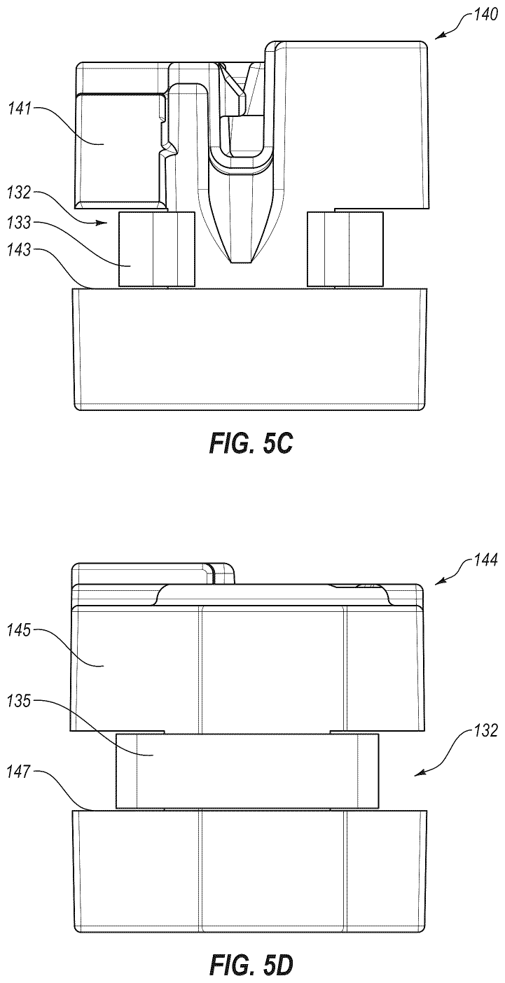

FIG. 5A is a perspective view of various embodiments of an injection slide 140, a return slide 144, and a rail 132 of a needle insertion mechanism 120. FIG. 5B is a top view of the injection slide 140, the return slide 144, and the rail 132 of FIG. 5A. FIG. 5C is a cross-section of the injection slide of FIGS. 5A and 5B. FIG. 5D is a back view of the return slide of FIGS. 5A and 5B. Referring to FIGS. 5A-5D, in various embodiments, the injection slide 140 includes an injection slide body 141 with one or more injection slide rail slots 143 formed therein, and the return slide 144 includes a return slide body 145 with one or more return slide rail slots 147 formed therein. In the various embodiments shown in FIGS. 5A-5D, each of the injection slide body 141 and the return slide body 145 includes an I-shaped cross section (cut perpendicular to the first and second directions), the I-shape defining the respective injection slide rail slots 143 and return slide rail slots 147.

In various embodiments, the rail 132 is formed as a single unitary structure and is generally formed with a U-shape (e.g., a narrow, elongated U-shape, without limitation). In some of these various embodiments, the rail 132 is formed from a single unitary piece of sheet metal bent into the U-shape. The rail 132 includes a bend 135 at the end of the U-shape with sides 133 extending from the bend 135. The sides 133 are configured to guide movement of the injection slide 140 and the return slide 144 in the first direction and the return slide 144 in the second direction (e.g., limiting the injection slide 140 and the return slide 144 to one degree of freedom, without limitation). The rail 132 may generally include a substantially rectangular cross-section. While assembled, each side 133 of the rail 132 extends through a respective injection slide rail slot 143 and a respective return slide rail slot 147, and the return slide 144 is positioned closer to the bend 135 than the injection slide 140.

In various embodiments, the rail 132 includes retention arms 137 with a retention arm 137 extending from an end of a respective side 133 distal to the bend 135. The retention arms 137 are configured to retain the injection slide 140 and the return slide 144 on the rail 132. The retention arm 137 is bent at an angle relative to the respective side 133. In the various embodiments of FIGS. 5A-5D, each retention arm 137 extends inward toward the other of the retention arms 137 with distal ends of the retention arms 137 closer together than distal ends of the sides 133 (distal relative to the bend 135). In various embodiments, for assembly, the sides 133 flex outward to provide clearance between ends of the retention arms 137 for the injection slide 140 and the return slide 144 to be received therethrough and for the sides 133 to be received within the injection slide rail slots 143 and the return slide rail slots 147. After which, the rail 132 returns to the original geometry and the inward extension of the retention arms 137 is sufficient to retain the injection slide 140 and the return slide 144 on the rail 132.

The rail 132 may be supported by a portion of the frame 126 or the chassis 104 (e.g., supported at the bend 135, without limitation).

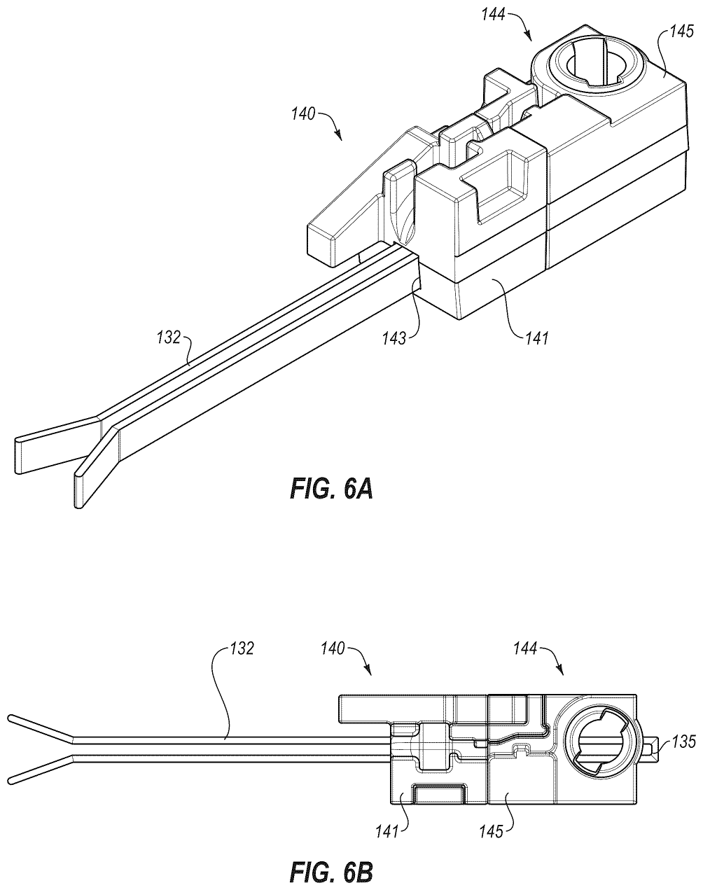

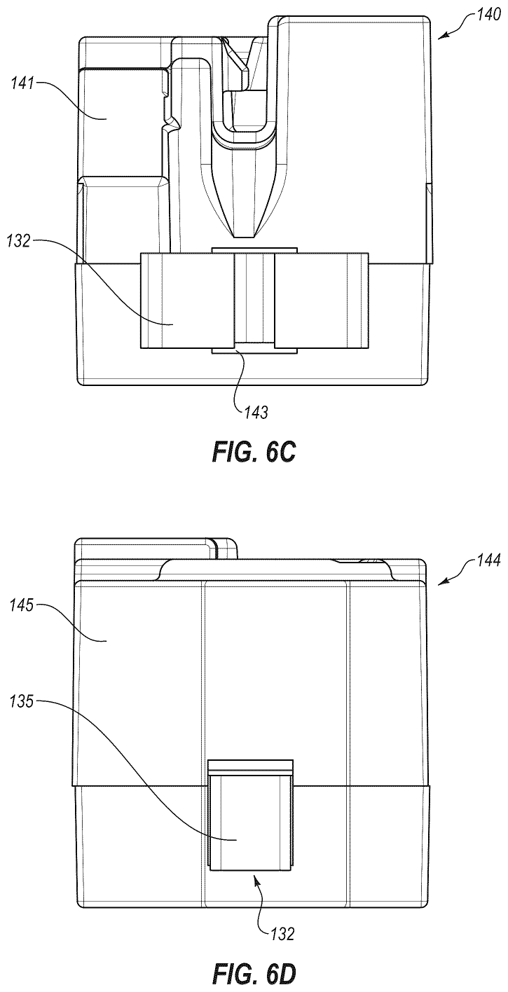

FIG. 6A is a cross-section of various embodiments of an injection slide 140 for a needle insertion mechanism 120. FIG. 6B is a top view of the injection slide 140 of FIG. 6A, the return slide 144, and the rail 132. FIG. 6C is a cross-section of the injection slide 140 of FIGS. 6A and 6B. FIG. 6D is a back view of the return slide 144 of FIGS. 6A and 6B. Referring to FIGS. 6A-6D, in various embodiments, the injection slide body 141 forms a single injection slide rail slot 143 therein, and the return slide body 145 forms a single return slide rail slot 147 therein. In the various embodiments shown in FIGS. 6A-6D, each of the injection slide body 141 and the return slide body 145 forms the injection slide rail slot 143 and the return slide rail slot 147 internally within the respective injection slide body 141 and return slide body 145.

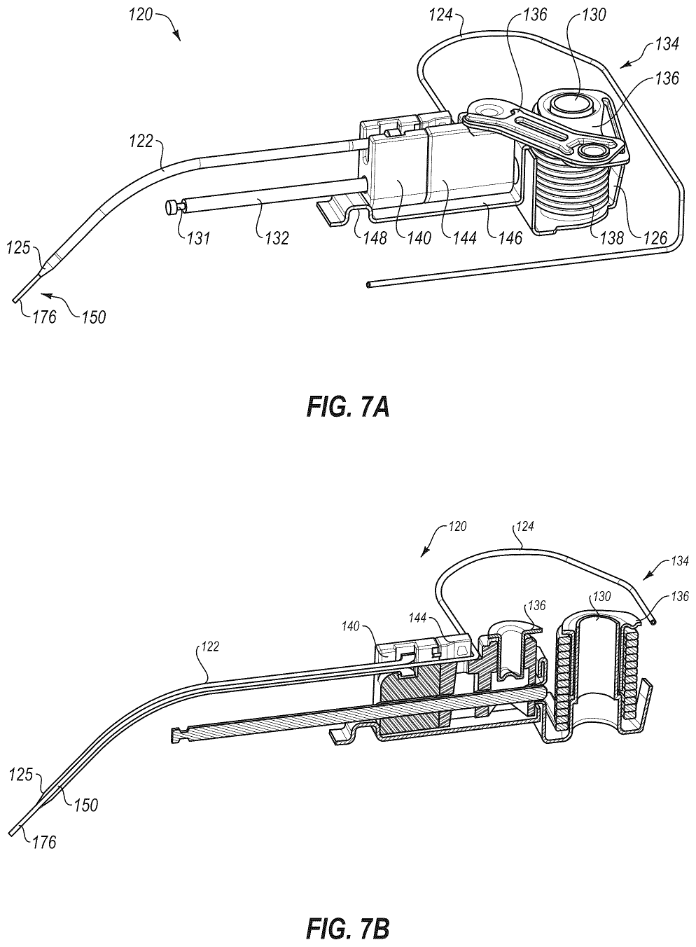

In various embodiments, the rail 132 is configured to be received in the single injection slide rail slot 143 and the single return slide rail slot 147 to guide movement of the injection slide 140 and the return slide 144 in the first direction and the return slide 144 in the second direction (e.g., limiting the injection slide 140 and the return slide 144 to one degree of freedom, without limitation). In these various embodiments, the rail 132 includes a monorail structure chosen from among a U-shape (e.g., a narrow, elongated U-shape, without limitation) with sides 133 positioned relatively close together (e.g., the sides 133 being adjacent or adjoining one another, without limitation) and a singular rod (e.g., a slender bar or solid wire, without limitation) (refer to FIGS. 7A-9 discussed below).

Similar to the various embodiments described above with regards to FIGS. 5A-5D, the U-shaped monorail configuration may be a single unitary structure. In some of these various embodiments, the rail 132 is formed of a single unitary piece of sheet metal that is bent into the U-shape. The rail 132 includes a bend 135 at the end of the U-shape with sides 133 extending from the bend 135. The rail 132 may generally include a substantially rectangular cross-section. While assembled, the sides 133 support each of the injection slide 140 and the return slide 144 together from within the single injection slide rail slot 143 and the single return slide rail slot 147, and the return slide 144 is positioned closer to the bend 135 than the injection slide 140.

Similar to the various embodiments described above with regards to FIGS. 5A-5D, in various embodiments, the rail 132 includes retention arms 137 with a retention arm 137 extending from an end of a respective side 133 distal to the bend 135. The retention arms 137 are configured to retain the injection slide 140 and the return slide 144 on the rail 132. The retention arm 137 is bent at an angle relative to the respective side 133. In the various embodiments of FIGS. 6A-6D, each retention arm 137 extends outward away from the other of the retention arms 137 with distal ends of the retention arms 137 further apart than distal ends of the sides 133 (distal relative to the bend 135). In various embodiments, for assembly, the sides 133 flex inward to provide clearance for ends of the retention arms 137 to be received within and pass through the injection slide rail slots 143 and the return slide rail slots 147. After which, the rail 132 returns to the original geometry and the outward extension of the retention arms 137 is sufficient to retain the injection slide 140 and the return slide 144 on the rail 132.

The rail 132 may be supported by a portion of the frame 126 or the chassis 104 (e.g., supported at the bend 135, without limitation).

FIG. 7A is a perspective view of a needle insertion mechanism 120 in a pre-deployed state, in accordance with various embodiments. FIG. 7B is a partial cross-section of the needle insertion mechanism 120 of FIG. 7A. As noted above, in various embodiments, the monorail structure of the rail 132 includes a singular rod (e.g., a slender bar or solid wire, without limitation) configured to support and guide movement of the injection slide 140 and the return slide 144 in the first direction and the return slide 144 in the second direction (e.g., limiting the injection slide 140 and the return slide 144 to one degree of freedom, without limitation). The rod may generally include a uniform shape (e.g., a substantially right circular cylinder shape or a substantially cuboid shape, without limitation) that includes a generally constant cross-sectional shape (e.g., a circular cross-sectional shape, a square cross-sectional shape, or a rectangular cross-sectional shape, without limitation). The rod may include a height/width or a diameter from about 1 millimeter to about 3 millimeters. In some of these embodiments, the rail 132 includes a connection feature 131 formed therein. The connection feature 131 is configured to connect the rail 132 to the chassis 104.

In various embodiments, the actuator 134 includes multiple actuator arms 136, and the spring 138 includes a spiral spring. In other various embodiments, the various embodiments of the rail 132 described with regards to FIGS. 5A-7B are combined with the actuator 134 described with regards to FIGS. 1-4B. Other combinations and configurations are also contemplated.

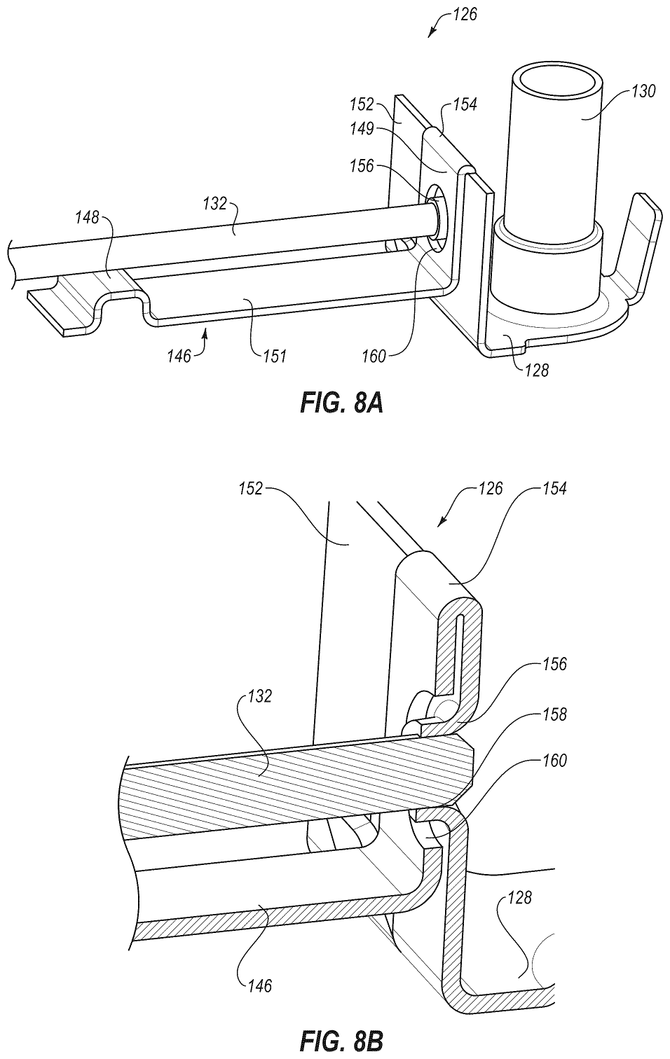

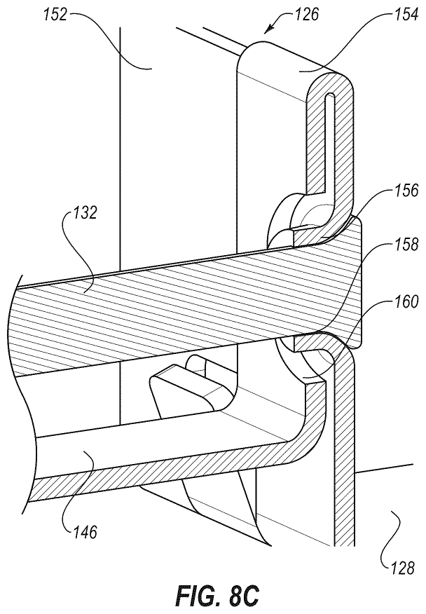

FIG. 8A is a perspective view of a frame 126 of the needle insertion mechanism 120 of FIG. 7A and FIG. 7B. FIG. 8B is a detailed cross-section of an embodiment of a portion of the frame 126 of FIG. 8A. FIG. 8C is a detailed cross-section of another embodiment of the portion of the frame 126 of FIG. 8A. In various embodiments, the frame 126 includes a frame body 128, a vertical wall 152, the rail 132, and a locking arm 146. In these various embodiments, the vertical wall 152 extends from the frame body 128 transverse to the first and second directions. The rail 132 is connected to the vertical wall 152 and extends in the first direction from the vertical wall 152 substantially perpendicular to the vertical wall 152.

The vertical wall 152 includes a drawn feature 156 defining a wall opening 158 formed therein. The drawn feature 156 transitions from a flat plate shape of the vertical wall to a hollow cylinder shape that defines the wall opening 158 and that is configured to receive an end of the rail 132. The drawn feature 156 is configured to hold an end of the rail 132 therein. In some of these various embodiments, the end of the rail 132 is held by an interference fit, held by a press fit, or floats within the drawn feature. In some of the various embodiments where the rail 132 includes the connection feature 131 to connect the rail 132 to the chassis 104, the end of the rail 132 received in the wall opening 158 formed by the drawn feature 156 may float therein. The connection feature 131 may be formed in the rail 132 at an end of the rail 132 distal to the vertical wall 152.

In some of these embodiments, the rail 132 includes a taper at an end thereof that expands a width/diameter of the rails 132 (e.g., a shape of a nail head, without limitation). During assembly, an opposite end of the rail 132 is fed through the wall opening 158 until the taper at the end of the rail 132 forms an interference fit with the drawn feature 156 to secure the rail 132 to the frame body 128.

In various embodiments, the frame 126 includes a locking arm 146. In some of these various embodiments, the locking arm 146, the vertical wall 152, and the frame body 128 are formed from a single unitary structure (e.g., formed from a single piece of sheet metal, without limitation). In these various embodiments, the locking arm 146 includes a wall connection 154, a first portion 149, a second portion 151, and a locking feature 148. The wall connection 154 forms a connection between the first portion 149 and the vertical wall 152 (e.g., about a 180-degree bend between the vertical wall 152 and the first portion 149, without limitation).

The first portion 149 extends substantially parallel to the vertical wall 152, and the second portion 151 extends from an end of the first portion 149 distal to the wall connection 154 in the first direction, substantially parallel to the rail 132. The first portion 149 includes a locking arm opening 160 formed therein. The locking arm opening 160 aligns with the drawn feature 156 and the rail 132 (e.g., substantially concentric to the drawn feature 156 and the rail 132, without limitation). In some of these various embodiments, the drawn feature 156 extends in the first direction at least partially through the locking arm opening 160.

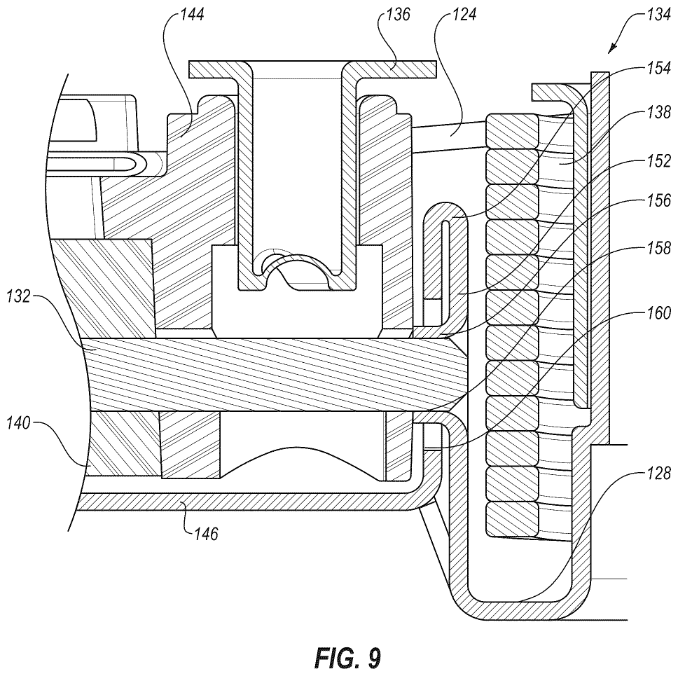

FIG. 9 is a cross-section of a portion of the needle insertion mechanism 120 of FIG. 7A and FIG. 7B. Referring to FIG. 9, in various embodiments, the vertical wall 152 and the end of the rail 132 received in the wall opening 158 formed by the drawn feature 156 are positioned adjacent to the spring 138. This configuration may allow the return slide 144 to retract to a position relatively close to the spring 138, resulting in an efficient deployment/retraction stroke by the actuator 134.

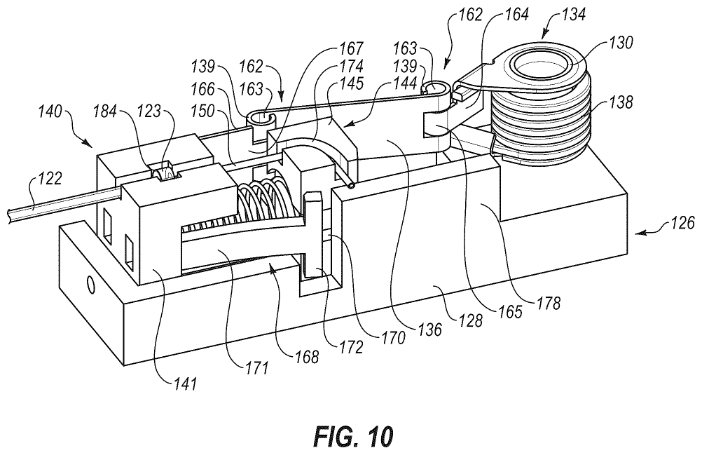

FIG. 10 is a perspective view of a needle insertion mechanism 120. Referring to FIG. 10, in various embodiments, the actuator 134 includes a hinged configuration, in accordance with various embodiments. In particular, the spring 138 includes a torsion spring and the actuator arm 136 is connected to each of the spring 138 and the injection slide 140 via a hinge 162. In various embodiments, the actuator arm 136 generally includes a plate shape (e.g., a thin, flat sheet or strip of metal or other material, without limitation).

In some of these various embodiments, the actuator 134 includes a spring connector 164 and a slide connector 166. The spring connector 164 forms the hinge 162 with the actuator arm 136 for connecting the spring 138 thereto. The spring connector 164 may be connected to and extend substantially tangentially from the spring 138 and the orientation thereof may be fixed relative to the spring 138. The slide connector 166 forms the hinge 162 with the actuator arm 136 for connecting the injection slide 140 thereto. The slide connector 166 may be connected to and extend from the injection slide 140 (e.g., from a side of the injection slide 140, without limitation) in the second direction and the orientation thereof may be fixed relative to the injection slide 140.

In some of these various embodiments, the actuator arm 136 includes one or more arm knuckles 139 at each end thereof, the spring connector 164 includes one or more spring connector knuckles 165 at an end distal to the spring 138, and the slide connector 166 includes one or more slide connector knuckles 167 distal to the injection slide 140. Each of the one or more arm knuckles 139, the one or more spring connector knuckles 165, and the one or more slide connector knuckles 167 includes a circular, hollow shape (e.g., a hollow cylinder or a sector of a hollow cylinder, without limitation) and may be referred to as a barrel, knuckle, loop, joint, curl, or node. Each hinge 162 is formed by aligning respective knuckles and inserting a pin 163 therethrough. In the various embodiments illustrated, the hinge 162 connecting the actuator arm 136 to the spring connector 164 is formed via two arm knuckles 139 aligned with a spring connector knuckle 165 and a pin 163 inserted therethrough, and the hinge 162 connecting the actuator arm 136 to the slide connector 166 is formed via two arm knuckles 139 aligned with a slide connector knuckle 167 and a pin 163 inserted therethrough.

In various embodiments, at least one of the injection slide 140 and the return slide 144 includes one or more features configured to connect the injection slide 140 and the return slide 144 together and maintain a relative position therebetween while in the pre-deployed state and during deployment of the cannula 122. The one or more features are configured to release the connection after deployment of the cannula 122 to facilitate retraction of the needle 150.

In some of these various embodiments, the one or more features include a connection slot 170 formed in the return slide body 145 (e.g., formed in a side of the return slide body 145, without limitation) and a connection arm 168 extending from the injection slide body 141 (e.g., from a side of the injection slide body 141, without limitation). The injection slide 140 may include the connection arm 168 (e.g., unitarily formed with the injection slide body 141 or integrally connected to the injection slide body 141, without limitation). The connection arm 168 includes a connection feature 172 that is configured to secure the injection slide 140 and the return slide 144 together (e.g., hold the return slide 144 adjacent to or adjoined to the injection slide 140, without limitation). In various embodiments, the connection feature 172 extends transverse to a connection arm body 171 of the connection arm 168 (e.g., the connection feature 172 extends from an end of the connection arm body 171 defining a T-shape therewith, without limitation). While the various embodiments illustrated in FIG. 10 show the injection slide 140 including the connection arm 168 and the return slide 144 including the connection slot 170, in other various embodiments, the return slide 144 includes the connection arm 168 and the injection slide 140 includes the connection slot 170.

In various embodiments, the frame 126 includes a retention wall 178 extending from the frame body 128 (e.g., unitarily formed with the frame body 128 or integrally connected to the frame body 128, without limitation). The retention wall 178 is configured to hold the connection feature 172 in position relative to the return slide 144 until the cannula 122 is deployed (e.g., positioned relative to the connection feature 172 to hold the connection feature 172 in position relative to the return slide 144 until the cannula 122 is deployed, without limitation).

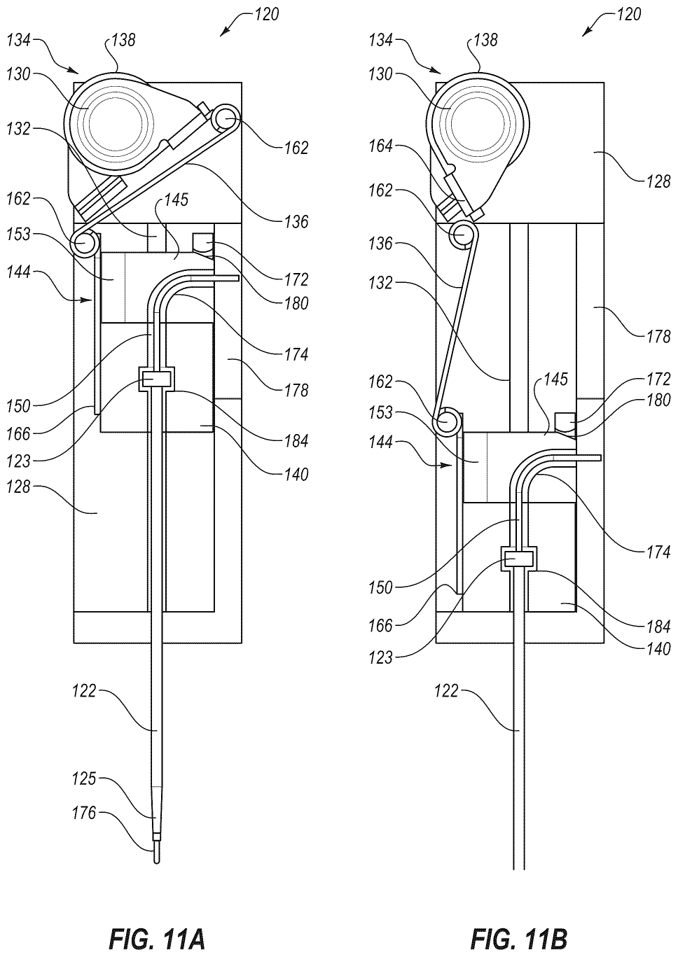

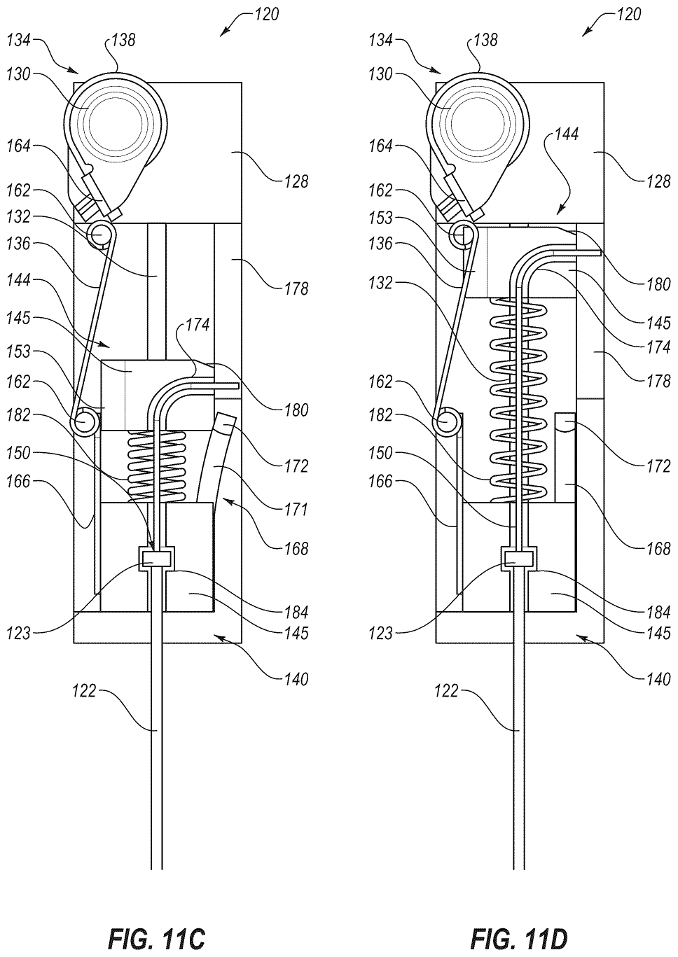

FIGS. 11A-11D illustrate the firing of the needle insertion mechanism 120 and various positions thereof during deployment of the cannula 122 and subsequent retraction of the needle 150. FIG. 11A is a top view of the needle insertion mechanism 120 of FIG. 10 in a pre-deployed state. FIG. 11B is a top view of the needle insertion mechanism 120 of FIG. 10 in a deployed state. FIG. 11C is a top view of the needle insertion mechanism 120 of FIG. 10 in a partially retracted state. FIG. 11D is a top view of the needle insertion mechanism 120 of FIG. 10 in a post-deployed/fully retracted state.

Referring to FIG. 11A, in the pre-deployed state, the spring 138 is in a wound state with sufficient potential energy to cause the injection slide 140 and the return slide 144 to fully deploy the cannula 122 and the needle 150, respectively. The actuator arm 136 is positioned adjacent to the spring 138 (e.g., the hinge 162 connecting the actuator arm 136 to the slide connector 166 positioned adjacent to the spring 138, without limitation) with the hinge 162 connecting the actuator arm 136 to the spring 138 rotated at a first angle relative to the spring connector 164. In various embodiments, the first angle is at least one-hundred and eighty degrees. The actuator arm 136 may be positioned substantially within a footprint of the frame body 128 (e.g., with one or more hinges 162 partially protruding beyond the footprint, without limitation).

In the pre-deployed state, the injection slide 140 and the return slide 144 are connected via the one or more features (e.g., the connection arm body 171 positioned within the connection slot 170, and the connection feature 172 positioned on an end of the return slide body 145 opposite the position of the injection slide body 141, without limitation).

In various embodiments, the injection slide 140 includes a cannular retention slot 184 formed in the injection slide body 141 and configured to retain a mating end 123 of the cannula 122 therein. The mating end 123 is distal to the insertion end 125 of the cannula 122 and may be radially larger than a remainder of the cannula 122.

In various embodiments, the return slide 144 includes a needle slot 174 formed in the return slide body 145. The retention needle slot 174 includes an arcuate shape (e.g., about a ninety-degree bend, without limitation) configured to retain a portion of the needle 150/tubing 124 therein and to cause the needle 150/tubing 124 to move therewith. While the injection slide 140 and the return slide 144 are connected, the needle tip 176 extends at least partially out from the insertion end 125 of the cannula 122.

The injection slide 140 and the return slide 144 are mounted on the rail 132. While the rail 132 illustrated in FIGS. 11A-11D includes a monorail, other rail configurations are also contemplated (e.g., any of the one or more rails 132 disclosed herein).

Referring to FIG. 11B, upon release, the actuator 134 causes the injection slide 140 and the return slide 144 to move to a deployed state (in a deployed position) resulting in deployment of the cannula 122 and the needle 150 with the needle tip 176 guiding the insertion end 125 of the cannula 122 into the user-body. In particular, the spring 138 unwinds, causing the actuator arm 136 to rotate relative to the spring 138 and relative to the injection slide 140 at the respective hinges 162, resulting in lateral movement of the injection slide 140 and the return slide 144 along the rail 132 from the pre-deployed state (pre-deployed position) to the deployed state (deployed position) in the first direction. In various embodiments, the spring 138 is configured to rotate between 90 to 135 degrees from the pre-deployed state to the deployed state. In various embodiments, the actuator arm 136 causes the injection slide 140 to move along the rail 132 in the first direction via the slide connector 166, and the return slide 144 moves with the injection slide 140 due to the connection therebetween formed by the one or more features (e.g., the connection arm 168 and the connection slot 170, without limitation). The angle between the actuator arm 136 and the spring connector 164 reduces from the first angle to a second angle (e.g., less the one-hundred and eighty degrees, less than ninety degrees, or less than forty-five degrees, without limitation). In various embodiments, the hinges 162 operate similar to an operation of a door hinge. In various embodiments, the hinges 162 lock or bind to prevent retraction or movement of the injection slide 140 once the injection slide 140 reaches the deployed state (deployed position). The movement of the hinges 162 may reduce the size of the overall movement paths of the components of the actuator 134, which may reduce the size for the needle insertion mechanism 120.

The release of the actuator 134, injection slide 140, and the return slide 144 caused by the locking mechanism (e.g., a locking flexure arm holding one of the spring 138 or the injection slide 140, without limitation) being caused to move (e.g., a wheel spins the locking flexure arm a predetermined amount, without limitation).

With the use of a rotary spring and hinges 162 controlling the movements of the actuator 134, friction between the actuator 134 and a cover of the automated medicament delivery device 100 may be reduced. Further, the hinges 162 may reduce upward movement of the actuator 134, which may reduce flexing or buckling within the cover and/or the chassis 104 of the automated medicament delivery device 100.

Referring to FIGS. 11C and 11D, in various embodiments, the needle insertion mechanism 120 includes a return spring 182 configured to separate the injection slide 140 and the return slide 144. In some of these various embodiments, the return spring 182 is positioned between the injection slide 140 and the return slide 144. The return spring 182 may be connected to each of the injection slide 140 and the 144 at respective ends thereof. In the pre-deployed state and until release of the connection between the injection slide 140 and the return slide 144, the return spring 182 is in a loaded/compressed state.

At or about full deployment of injection slide 140 and the insertion end 125 of the cannula 122 into the user-body (e.g., the needle insertion mechanism 120 being in the deployed state), the one or more features release the connection between the injection slide 140 and the return slide 144, and the return spring 182 causes the return slide 144 to retract in the second direction to a post-deployed state (post-deployed position) resulting in retraction of the needle 150, and in particular, the needle tip 176 from a protruding position (e.g., protruding from the insertion end 125) to a retracted position within the cannula 122.

In some of these various embodiments, the retention wall 178 is configured to hold the connection arm 168 including the connection feature 172 in position relative to the return slide 144, the return slide 144 includes a chamfer 180 formed in the return slide body 145, and the connection arm body 171 is configured to flex outward. A length of the retention wall 178 is configured such that the connection arm 168 including the connection feature 172 clears the retention wall 178 at or about the injection slide 140 and the return slide 144 reaching the deployed position, resulting in release of the connection arm 168 including the connection feature 172. The connection feature 172 adjoins the chamfer 180, and upon clearing the retention wall 178, the force applied by the return spring 182 to the return slide 144 causes the connection arm body 171 to flex responsive to the interaction between the connection feature 172 and the chamfer 180, releasing the connection between the injection slide 140 and the return slide 144 and facilitating retraction of the return slide 144 and the needle 150 connected thereto. This passive release of the connection between the injection slide 140 and the return slide 144 may reduce the overall complexity of the needle insertion mechanism 120 and may improve reliability of the needle insertion mechanism 120.

As illustrated in FIG. 11D, in the post-deployed state, the injection slide 140 remains in the deployed position, at a distal end of the rail 132 relative to a position of the spring 138, the cannula 122 is in a deployed position with the insertion end 125 of the cannula 122 positioned within the user-body (e.g., positioned for subcutaneous delivery of a medicament, without limitation), the return slide 144 is in a retracted position separated from the injection slide 140 at a proximal end of the rail 132 relative to the position of the spring 138, and the needle tip 176 is in a retracted position within the cannula 122 between the insertion end 125 and the mating end 123.

In various embodiments, the return slide 144 includes a cutout 153 formed in the return slide body 145. The cutout 153 is configured to prevent interference or contact between the return slide 144 and the actuator arm 136 (refer to FIGS. 10, 11C, and 11D). The cutout aligns with a vertical position of the actuator arm 136 and the spring 138 and is positioned on a side of the return slide 144 opposite a position of an end of the needle slot 174, defining an exit for the needle 150/tubing 124 from the needle insertion mechanism 120 (e.g., toward the reservoir 108, without limitation).

In the detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

The illustrations presented herein are not meant to be actual views of any particular method, system, device, or structure, but are merely idealized representations that are employed to describe the embodiments of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawing could be arranged and designed in a wide variety of different configurations. Thus, the description of various embodiments is not intended to limit the scope of the present disclosure but is merely representative of various embodiments. While the various aspects of the embodiments may be presented in drawings, the drawings are not necessarily drawn to scale unless specifically indicated.

In the Brief Summary and in the Detailed Description, the claims, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

The embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

As used herein, the term “substantially” in reference to a given parameter, property, or condition means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or even at least about 99% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the terms “adapted,” “configured,” and “configuration” refers to a size, a shape, a material composition, a material distribution, orientation, and arrangement of at least one feature (e.g., one or more of at least one structure, at least one material, at least one region, at least one device) facilitating use of the at least one feature in a pre-determined way.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

What is claimed is:

1. An automated medicament delivery device or system, comprising:
a cannula including an insertion end;
a needle including a needle tip; and
a cannula insertion mechanism comprising:
an injection slide configured to cause movement of the insertion end;
a return slide configured to cause movement of the needle tip;
one or more rails;
an actuator configured to cause movement of the injection slide and the return slide along the one or more rails, the actuator comprising:
a spiral spring; and
an actuator arm including a first end rotatably connected to the return slide and a second end rotatably connected to an external end of the spiral spring.

2. The automated medicament delivery device or system of claim 1, wherein, in a pre-actuated state, the spiral spring is in a wound condition, and upon release thereof is configured to cause the return slide and the injection slide to move in a first direction along the one or more rails to a deployed state via the single actuator arm, resulting in insertion of the insertion end and the needle tip into a user-body.

3. The automated medicament delivery device or system of claim 2, wherein the spiral spring is configured to further uncoil and cause the return slide to move away from the injection slide in a second direction, opposite the first direction, along the one or more rails to a post-deployed state via the single actuator arm, resulting in withdrawal of the needle tip from the user-body.

4. The automated medicament delivery device or system of claim 1, wherein the one or more rails is a single rail formed as a single unitary structure and includes a U-shape.

5. The automated medicament delivery device or system of claim 4, wherein the injection slide includes an injection slide slot formed therein and the return slide includes a return slide slot formed therein, each of the injection slide slot and the return slide slot configured to receive a side of the U-shape of the single rail.

6. The automated medicament delivery device or system of claim 5, wherein each of the injection slide slot and the return slide slot is configured to receive both sides of the U-shape therein.

7. The automated medicament delivery device or system of claim 4, wherein the single rail is formed from a single unitary piece of sheet metal bent into the U-shape.

8. The automated medicament delivery device or system of claim 4, wherein the single rail includes sides, a bend connecting the sides together, and retention arms configured to retain the injection slide and the return slide on the single rail, each of the retention arms extending from a respective side of the sides.

9. The automated medicament delivery device or system of claim 8, wherein the retention arms extend in the sides extend toward one another with distal ends of the retention arms closer together than distal ends of the sides or extend away from one another with the distal ends of the retention arms further apart than the distal ends of the sides.

10. An automated medicament delivery device or system, comprising:
a cannula including an insertion end;
a needle including a needle tip; and
a cannula insertion mechanism comprising:
a frame comprising:
a vertical wall including a drawn feature formed therein, the drawn feature defining a wall opening; and
a single rail formed as a single unitary structure, the single rail including a first end received in the wall opening;
an injection slide configured to move the insertion end therewith, the injection slide including an injection slide slot formed therein, the injection slide slot configured to receive the single rail;
a return slide configured to move the needle tip therewith, the return slide including a return slide slot formed therein, the return slide slot configured to receive the single rail; and
an actuator configured to cause movement of the injection slide and the return slide along the single rail.

11. The automated medicament delivery device or system of claim 10, wherein the single rail extends substantially perpendicular to the vertical wall.

12. The automated medicament delivery device or system of claim 10, wherein the drawn feature transitions from a flat plate shape of the vertical wall to a hollow cylinder shape that defines the wall opening and that is configured to receive the first end of the single rail.

13. The automated medicament delivery device or system of claim 10, wherein the first end of the single rail is held in the wall opening via an interference fit.

14. The automated medicament delivery device or system of claim 10, wherein the single rail includes a connection feature formed in a second end of the single rail distal to the vertical wall configured to connect the single rail to a chassis of the automated medicament delivery device.

15. The automated medicament delivery device or system of claim 14, wherein the first end is configured to float within the wall opening while the second end is connected to the chassis.

16. The automated medicament delivery device or system of claim 15, wherein the frame further includes a locking arm configured to retain the injection slide and the return slide in a pre-deployed state.

17. The automated medicament delivery device or system of claim 16, wherein the frame is integrally formed as a single unitary structure, the locking arm including a first portion and a second portion, the first portion connected to the vertical wall via a wall connection and extending substantially parallel to the vertical wall, the second portion extending from the first portion substantially parallel to the single rail.

18. The automated medicament delivery device or system of claim 10, wherein the actuator includes a spiral spring.

19. The automated medicament delivery device or system of claim 18, wherein the actuator further including a single actuator arm including a first actuator end rotatably connected to the return slide and a second actuator end rotatably connected to an external end of the spiral spring.

20. The automated medicament delivery device or system of claim 19, wherein, in a pre-actuated state, the spiral spring is in a wound condition, and upon release thereof is configured to cause the return slide and the injection slide to move in a first direction along the single rail to a deployed state via the single actuator arm, resulting in insertion of the insertion end and the needle tip into a user-body.

21. The automated medicament delivery device or system of claim 20, wherein the spiral spring is configured to further uncoil and cause the return slide to move away from the injection slide in a second direction, opposite the first direction, along the single rail to a post-deployed state via the single actuator arm, resulting in withdrawal of the needle tip from the user-body.
